(19) United States
(12) Patent Application Publication
(10) Pub. No.: US 2010/0233306 A1
(43) Pub. Date: Sep. 16, 2010

(54) METHODS AND COMPOSITIONS FOR WEIGHT LOSS USING GARCINIA INDICA FRUIT PUREE

(76) Inventor: Sevanti Mehta, Houston, TX (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/405,045

(22) Filed: Mar. 16, 2009

(51) Int. Cl. A61K 36/38; A61K 31/194; A61P 3/04; A61K 131/00

(52) U.S. Cl. 424/777; 514/574

(57) ABSTRACT

Methods for increasing weight loss in a mammal in need thereof are provided. In one embodiment the method comprises administering to a mammal a weight loss effective amount of Garcinia indica fruit purée. In another embodiment of the present invention, a method of administering to a mammal a weight loss effective amount of naturally occurring HCA, cyanidin-3-sambubioside and cyanidine 3-glucoside from Garcinia indica fruit purée is provided. In another embodiment, a method for providing a stabilized hydroxycitric acid composition comprising hydroxycitric acid and anthocyanins to provide a very pure, stabilized preparation that is substantially tasteless for optimal use in a variety of foods items which includes treating an aqueous extract of Garcinia indica fruit with a liquid quaternizing agent such as a trialkylamine in which the alkyl groups are octyl, caprylyl, isooctyl, lauryl or decyl.

### BACKGROUND

1. Field

The present disclosure generally relates to methods for weight loss using Garcinia indica. More specifically, the present disclosure is related to method for providing weight loss to a mammal using a composition of Garcinia indica fruit purée and methods of stabilizing the natural HCA in such compositions.

2. Background

Use of G. indica fruit has been reported during the 16^{th} century (Mathew, 1997). Renaissance Portuguese Jewish physician and naturalist, Garcia DeOrta in 1563 reported the knowledge of G. indica fruit to the Portuguese of Goa (India) by the name Brindones (Watt, 1890). In India, G. indica trees are found in the tropical rain forest of Western Ghats from southward in Karnataka and also in southern parts of Maharashtra as well as in West Bengal and Assam. The tree flowers during November to February and fruits ripen in April-May. The ‘kokam’ of commerce is prepared by sun drying the rind of the ripe fruits after repeated soaking it in the juice of the pulp and sometime after treatment with salt (salted kokum) (Krishnamurthy et al., 1982). In Maharashtra state of India, commercial processing of kokam is well organized and serves as an additional source of income to farmer (Kruijssen, 2008). Among the varieties traded in India, Lonaval kokam, Pakali kokam, Khanee kokam and Khoba kokam are well known (Raju and Reni, 2001). Although no reliable information on the amount of G. indica fruit production is available, in the Kokan region of Maharashtra about 4000 tonnes are produced. One survey report has indicated that Western Ghats in India has over 1.5 million trees of G. indica (Sampathu and Krishnamurthy, 1982).

Garicia DeOrta mentioned that the fruit has a pleasant, though sour, taste and that the fruit serves to make vinegar (Hedrick, 1919). During the end of the 18^{th} century, fruit of G. indica was employed as anti-scorbutic in the Bombay Army, one of the three presidencies of the Empire of India within the British Empire. The fruit has an agreeable flavor and has long been esteemed as an article of diet (Watt, 1890). As such G. indica dried fruit rind (known as ‘kokam’) is an Indian spice used in many parts of the country for centuries. In India, it is commonly used to prepare several vegetarian and non-vegetarian “curry” preparations, including the popular “solkadhi” (kokam curry), a popular everyday food for each household in Konkan region (Maharashtra, India) (Raju and Reni, 2001; Mishra et al., 2006). The use of G. indica fruit extract is considered to be superior to tamarind for the preparation of acidulous drinks. The fruit of G. indica is regarded as underutilized species that is of importance in India. As G. indica fruit is used as a replacement for tamarind in the food preparation and about 10 g tamarind/person may be consumed. Hence it is likely that approximately 10 g of G. indica fruit or its extract is ingested daily. In a clinical study in children, the dose of 10 g tamarind per day was employed as safe dose (Khandare et al., 2002).

The fruits are steeped in sugar syrup to make ‘Amrutkokam’, a popular soft drink consumed during summer time (Kirtikar and Basu, 1984; Peter, 2001; Wildman, 2001). Kokam is commonly utilized as a garnish in Indian cuisine and is considered as an essential ingredient to a tasty local fish curry. In Maharashtra (a state in India), it is used as a slightly sour spice in recipes that yields peculiar taste and dark red color. It is a preferred substitute for tamarind in curries and other dishes from southern regions of India. The fact that G. indica fruit extract is in use for centuries and has been used in the production of food products prior to Jan. 1, 1958, supports its accepted safety. Available information demonstrates that G. indica fruit has a substantial history of consumption as a food by a significant number of consumers.

(−)-Hydroxycitric acid (HCA) is widely used as an ingredient for nutritional supplements aimed at reduction of food intake, appetite and body weight. However, studies on the effects of HCA in humans are controversial. (−)-Hydroxycitric acid (HCA) has been reported to cause weight loss in humans without stimulating the central nervous system (Clouatre and Rosenbaum, 1988). HCA is often derived from the fruit rinds of Garcinia indica, which exhibits a distinctive sour taste and has been used for culinary purposes in Southern Asia for centuries to make meals more “filling”, and has been reported to reduce food intake in experimental animals, suggesting its role in the treatment of obesity (Sergio, 1988; Sullivan et al., 1974). HCA is a competitive inhibitor of ATP-citrate lyase, an extra-mitochondrial enzyme involved in the initial steps of de novo lipogenesis (Sullivan et al., 1983; Sergio, 1988; Sullivan et al., 1974). Consequently, HCA reduces the transformation of citrate into acetyl coenzyme A, a step necessary for the formation of fatty acids in the liver. In addition, there is increased production of hepatic glycogen in the presence of HCA, which may activate glucoreceptors leading to a sensation of fullness and reduced appetite (Lowenstein, 1971; Triscari and Sullivan, 1984). Earlier successful animal trials (Sullivan et al., 1974) suggest that the human dose of HCA (as a salt) typically recommended in dietary supplements and used in previous clinical trials (1,500 mg HCA/day) is sub-optimal. Several publications have reported the efficacy of HCA in weight management (Ramos et al., 1995) although the results have been controversial and often contradicted by other reports and studies. Additionally, at relatively high concentrations, HCA can have negative side-effects, that are undesirable. More importantly, although HCA has been shown to provide some weight loss in certain instances, the results have been contradictory and did not provide the significant weight loss consumers are looking for. Even more importantly, in an effort to stabilize HCA in solution and in various compositions, HCA is almost always provide as an HCA salt, which is not as effective or desirable as naturally occurring HCA and causes various undesirable side effects.

A variety of methods of extracting HCA from the Garcinia fruit have been investigated. It has been found, however, that the free acid form of HCA is unstable, forming lactones which generally do not possess the desired bioactivity. Since the liquid form of free HCA tends to be unstable during storage, it has not been considered to be the optimal form for incorporation of HCA in food products. Therefore, it is generally thought that compositions which incorporate the free HCA in liquid form will not provide the full benefit of the functional product (i.e., HCA) in the final preparation.

Conventionally, the problem of instability of HCA has been addressed by preparing various salts of HCA, such as, for example, Li, Na, K, Rb, Cs, Fr, Be, Mg, Ca, Sr, Ba and Ra hydroxycitric acid salts, or double metal salts. The salt forms of HCA are generally more stable than free HCA and avoid or deter lactone formation. The calcium, magnesium, potassium and sodium salts of hydroxycitric acid have been used for increasing a person's glucose metabolism. The use of HCA salts in foods and drinks has been limited, however, by several drawbacks. Some of the HCA salts are excessively hygroscopic which contributes to poor keeping qualities and complicates handling of the material during commercial manufacturing. Some salts, such as calcium HCA salts, are not very soluble in water. This severely limits their applicability for use in drinking water, beverages, ice cream, candies and food. In many cases, the assay of HCA is too low for the product to fulfill the intended use.

Most products containing calcium salts of HCA are able to deliver only a maximum assay of 60% HCA, thereby limiting the total availability of HCA. While the potassium salt of HCA is more soluble than the calcium salt, the potassium salt imparts a strong pungent taste of potassium to the product. This can severely limit the amount of potassium hydroxycitrate that can be incorporated into a food or drink to levels which are below the amount needed to achieve the desired functional effects of the HCA in the recipient. The assay of HCA in conventional potassium hydroxycitrate preparations is also typically low, at 60% or less. Another drawback to utilization of HCA salts in foods, drinks and dietary aids is that conventional manufacturing methods tend to be attractive commercially due to the number of cumbersome steps and the handling problems arising from the hydroscopic nature of certain salts.

An even greater hindrance to the availability and use of HCA and its salts is that many HCA extraction procedures and HCA salt manufacturing methods employ organic or inorganic polar or non-polar solvents that can leave toxic residues in the product, or which produce other undesirable residues (e.g., high levels of chloride or oxalic acid residues) that are detrimental to the taste, color or fragrance of the HCA salt product. For example, some HCA extraction methods call for extracting the HCA in acetone. Potentially harmful or toxic residues of chemicals such as acetone and solvents used during manufacture are a particular concern in HCA compositions intended for human consumption.

Accordingly, there exists a need for stable HCA-containing compositions that are suitable for use as consumables such as drinking water, beverages, nutraceuticals, power bars, ice cream, and the like and are useful as diet aids to help with weight reduction. There also exists a need for a more effective completely natural and safe weight loss method and related composition that does not raise any safety concerns or unwanted side-effects. The present invention provides these and other benefits and advantages.

### SUMMARY

The present invention is direct to methods for increasing weight loss and improving related health factors in a mammal. In one embodiment of the present invention, a method for increasing weight loss in a mammal in need of such effect is provided. The method includes administering to a mammal a weight loss effective amount of Garcinia indica fruit purée.

In another embodiment of the present invention, a method for administering to a mammal a weight loss effective amount of naturally occurring HCA and anthocyanins from Garcinia indica fruit purée is provided.

In yet another embodiment of the present invention, a method of administering to a mammal a weight loss effective amount of naturally occurring HCA, cyanidin-3-sambubioside and cyanidine 3-glucoside from Garcinia indica fruit purée is provided.

In one aspect of the present invention, 1000-2000 milligrams of HCA and 300-1200 milligrams of anthocyanins from Garcinia indica fruit purée are provided to the mammal daily.

In another aspect of the present invention, 250 ml to 1000 ml, and more specifically, 500 ml of Garcinia indica fruit purée, is provided to the mammal daily.

In another aspect of the present invention, 1000 mg to 2000 mg, and more specifically, 1500 mg to 2000 mg of natural HCA from Garcinia indica fruit purée and 50 mg to 500 mg, and more specifically, 100 mg to 300 mg of cyanidin-3-sambubioside and cyanidine 3-glucoside from Garcinia indica fruit purée and is provided to the mammal daily.

In yet another aspect of the present invention, the effective amount decreases fat in the mammal.

In yet another aspect of the present invention, the effective amount decreases systolic and diastolic blood pressure in the mammal.

In yet another aspect of the present invention, the effective amount decreases triglyceride levels in the mammal.

In yet another aspect of the present invention, the effective amount decreases blood glucose levels in the mammal.

In yet another aspect of the present invention, the effective amount decreases total cholesterol levels in the mammal.

In yet another aspect of the present invention, the effective amount increases HDL cholesterol levels in the mammal.

In yet anther embodiment, a method of making a stabilized naturally occurring hydroxycitric acid composition is provided. The method comprises extracting Garcinia fruit containing hydroxycitric acid with an aqueous liquid comprising at least 8 mg/ml anthocyanin per 100 ml of liquid, to provide an aqueous extract and mixing the aqueous extract with a liquid quaternizing agent to yield a quaternizing agent extract that contains 1000 mg to 2500 mg of naturally occurring hydroxycitric acid.

In yet another aspect of the present invention, the quaternizing agent is a trialkylamine comprising an alkyl chosen from the group consisting of octyl, caprylyl, isooctyl, lauryl and decyl.

In yet another aspect of the present invention, the trialkylamine is tricaprylylamine.

In yet another aspect of the present invention, an anthocyanin-stabilized hydroxycitric acid composition prepared according to the methods described herein is provided.

In yet another aspect of the present invention, the stabilized naturally occurring hydroxycitric acid composition described herein is mixed with an edible food material.

In yet another aspect of the present invention, the stabilized naturally occurring hydroxycitric acid composition described herein is mixed with a drinkable liquid.

In yet another aspect of the present invention, the stabilized naturally occurring hydroxycitric acid composition described herein is mixed with an excipient.

In yet another embodiment of the present invention, a method of reducing body weight comprising administering to a mammal in need of such weight reduction an effective amount of the compositions described herein are provided.

### DRAWINGS

The above-mentioned features and objects of the present disclosure will become more apparent with reference to the following description taken in conjunction with the accompanying drawings wherein like reference numerals denote like elements and in which:

FIG. 1 shows the chemical structures of HCA and anthocyanin (cyanidin-3-sambubioside) and cyanidin 3-glucoside (CG3).

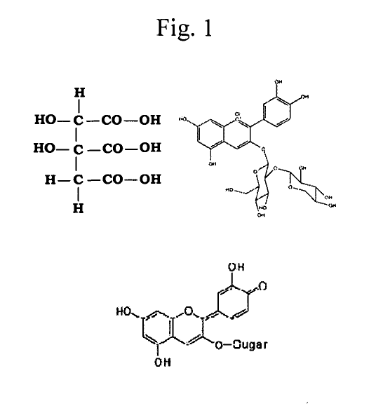

### DETAILED DESCRIPTION

Garcinia indica is a slender evergreen tree with drooping branches that thrive prolifically on the Indian subcontinent and in western Sri Lanka (Jena et al., 2002). Its fruits are globose or spherical, 1-1.5 inch diameter, dark purple when ripe and enclosing five to eight large seeds. The dried fruit rind of G. indica is commonly used, particularly in India, as a flavoring agent and carminative. The seeds of the fruit have edible fat, commercially known as Kokam butter. The fruit of G. indica has an agreeable flavor and a sweetish acid taste (Pruthi, 2001). Fruit from this plant is included in the United States Department of Agriculture's inventory of perennial edible fruits of the tropics (Martin et al., 1987).

Fruit extract of G. indica is a red, sparkling, free flowing liquid with characteristic odor or taste. The primary constituents of the extract are thought to be anthocyanins and (−)-HCA. General descriptive characteristics of G. indica fruit extract are summarized in Table 1 below.

TABLE 1
General descriptive characteristics of Garcinia indica Extract

| Parameter | Description |
|---|---|
| Botanical source | Garcinia indica |
| Botanical family | Clusiaceae (Guttiferae) |
| Plant part used | Dried fruit rind |
| Synonyms | Indian berry, Kokam, Kokum, Red mango |
| Appearance | Liquid |
| Color | Red, sparkling, free flowing liquid |
| Odor | Characteristic, sour |
| Taste | Characteristic |
| Principal ingredients | Anthocyanins; (−)-HCA |

The chemical structure of anthocyanins comprises a multi-ring system with positively charged hetero oxygen. The general structure of the anthocyanin nucleus can be represented as follows:

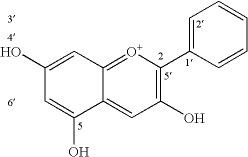

Certain substitutions of the general structure and the corresponding names of the resulting compounds are listed in Table 1.

TABLE 1
Representative Anthocyanin Compounds

| Substituent | Name |
|---|---|
| 4′ = OH | pelarigonidin |
| 3′, 4′ = OH | cynidin |
| 3′, 4′, 5′ = OH | delphinidin |
| 4′ = OH, 3′ = OMe | peonidin |
| 4′, 5′ = OH, 3′ = | peturidin |
| OMe | |
| 4′ = OH, 3′, 5′ = | malvidin |
| OMe | |

To determine the efficacy of a 500 ml daily dosage of Garcinia indica fruit purée (formulation) on weight loss and other related health factors, a controlled study was designed to examine the efficacy of in sixty human volunteers. Effects of formulation were investigated on body weight, BMI, lipid profiles, blood glucose, oxidative stress, hepatic and renal toxicities markers.

Participants

Sixty obese or overweight subjects were recruited for the eight-week study. Based on physical examination and laboratory screening tests, all diabetics as well as pregnant and lactating women were excluded. None of the participants took any weight-reducing medication nor followed any specific diet for the duration of the trial period. Of the 60 subjects, five (8.33%) were male and 55 (91.67%) were female. The age range was 28-62 (mean age=45.28) with a body mass index (BMI) ranging from 30.0 to 49.10 kg/m2 (BMI requirement was greater than ≧30kg/m2). The purpose, nature and potential risks of the study were explained to the patients, and all gave their written informed consent before participation. The Cameroon National Ethics Committee approved the protocol. The study was conducted in accordance with the Helsinki Declaration (1983 version).

Study Design and Intervention

The study was a randomized, double-blind, placebo-controlled design. The participants were randomly divided into two equal groups (n=30): HCA-A and HCA-B. Subjects in both groups were given 250 ml of an HCA drink divided into two equal doses 30-60 minutes before breakfast or lunch and dinner (two total daily doses) for eight weeks. The 60 subjects were examined once a week during the eight-week study period, and their body weight, percent body fat, and waist circumference recorded. Fasting blood samples were taken at baseline and at two, four and eight weeks. In addition to these physiological measurements, the patients' subjective impressions of their well-being (e.g., increased/decreased appetite, dizziness, gastrointestinal pains, etc.) were solicited and recorded at every visit. Although no major dietary changes or exercises were suggested, the subjects were queried re their physical activity and food intake.

Anthropometric Measurements

Body weight, percent body fat, and waist circumference were assessed at each visit with a Tanita™ BC-418 Segmental Body Composition Analyzer/Scale that uses bio-electrical impedance analysis for body composition analysis. Height was measured with a Harpended™ stadiometer, which measures the length of curved line staffage to the nearest 0.5 cm. Participants (12 hour fasted) were encouraged to wear light clothing before measurements were taken. The waist circumference was measured by soft, non-stretchable plastic tape on the narrowest and widest parts of the trunk.

Serological and Laboratory Methods

Blood samples were collected into heparinized tubes after a 12-hour overnight fast at the beginning of the study and after four, eight, and ten weeks of treatment. The concentrations of total cholesterol, HDL cholesterol, and fasting blood glucose in plasma were measured using commercial diagnostic kits from SIGMA Diagnostics, St. Louis, Mo., USA. Oxidative stress, hepatic and renal toxicity were measured using appropriated methods.

Statistical Analysis

The data for each parameter was summarized (n, mean, and standard deviation) for Week 0 (Initial) and Weeks 4, 8, and 10 and for the intra-group percent differences (Initial vs. Week 2, week 4 and Week 8).

Experimental Results

We noticed a non-significant change in body weight, body fat, and metabolic parameters after eight weeks of treatment with the control HCA-B group whereas the control experimental HCA-A groups, which contained only naturally occurring HCA and no HCA salts, showed significant weight loss. It is important to note that the weight loss shown by the HCA-A group was significantly more than the weight loss shown by other HCA products, which contain HCA salts, and at a much lower dose of HCA, which is not only more cost effective but avoid the many side effects often associated with higher levels of HCA consumption and use of HCA salts.

Table 6 below shows the results of our experiments showing the effect of hydroxycitrate (HCA) drink on anthropometric measurements after eight weeks of treatment as indicated herein. As the data demonstrates, Group HCA (A) showed statistically significant reduction in weight, BMI, fat, and waist size when compared to the control HCA (B). The active HCA drink taken by the HCA (A) experimental group appears to have synergistic qualities, at least partially attributable to the naturally occurring anthocyanins present with the natural HCA (as opposed to HCA salts) in the purée of Garcinia indica fruit purée, which provides significant weight and fat loss when compared to any known HCA product or supplement at a significantly lower HCA dosage.

TABLE 6

| | Treatment period | HCA (A) | HCA (B) |
|---|---|---|---|
| Weight (Kg) | week-0 | 101.84 ± 14.51 | 99.70 ± 10.28 |
| | week-2 | 98.46 ± 14.81 | 98.39 ± 9.66 |
| | week-4 | 96 ± 14.65 | 97.90 ± 10.19 |
| | week-8 | 95.78 ± 14.89 | 97.85 ± 10.18 |
| BMI (kg/m²) | week-0 | 36.76 ± 4.76 | 35.00 ± 4.16 |
| | week-2 | 34.02 ± 4.38 | 34.56 ± 4.11 |
| | week-4 | 33.27 ± 4.41 | 34.55 ± 4.13 |
| | week-8 | 32.28 ± 4.51 | 34.48 ± 4.10 |
| Fat (%) | week-0 | 44.32 ± 6.91 | 42.48 ± 6.14 |
| | week-2 | 41.93 ± 7.12 | 41.69 ± 6.35 |
| | week-4 | 39.81 ± 6.57 | 40.85 ± 6.10 |
| | week-8 | 37.08 ± 6.16 | 40.03 ± 6.19 |
| Waist size (cm) | week-0 | 108.45 ± 9.32 | 97.39 ± 7.12 |
| | week-2 | 101.72 ± 9.69* | 99.85 ± 9.53 |
| | week-4 | 97.34 ± 10.18* | 96.52 ± 7.98 |
| | week-8 | 95.55 ± 9.28** | 96.59 ± 6.51 |

*p < 0.001 compared to week-0

Table 7 below shows the effects of formula drink on blood pressure and metabolic parameters after eight weeks of treatment.

TABLE 7

| | Treatment | | |
|---|---|---|---|
| | period | HCA (A) | HCA (B) |
| Systolic Blood | week-0 | 143.30 ± 24.11 | 133.31 ± 21.18 |
| pressure (mmHg) | week-2 | 140.04 ± 18.12 | 134.00 ± 18.13 |
| | week-4 | 130.73 ± 16.53** | 131.63 ± 16.31 |
| | week-8 | 133.91 ± 19.80 | 130.40 ± 19.36 |
| Diastolic Blood | week-0 | 95.17 ± 15.19 | 90.63 ± 12.55 |
| pressure (mmHg) | week-2 | 93.21 ± 16.81 | 89.59 ± 13.91 |
| | week-4 | 89.17 ± 14.95 | 89.59 ± 12.38 |
| | week-8 | 92.30 ± 19.70 | 87.27 ± 10.99 |
| Blood glucose | week-0 | 105.38 ± 16.56 | 101.86 ± 11.60 |
| (mg/dL) | week-2 | 101.90 ± 35 | 99.19 ± 9.79 |
| | week-8 | 92.71 ± 17.75 | 99.19 ± 9.79 |
| Triglycerides | week-0 | 145.07 ± 70.20 | 146.60 ± 94.63 |
| (mg/dL) | week-2 | 135.89 ± 28.23* | 141.19 ± 22.18* |
| | week-8 | 120.59 ± 35.86* | 144.58 ± 37.64* |
| Total Cholesterol | week-0 | 196.73 ± 48.71 | 180.45 ± 57.60 |
| (mg/dL) | week-2 | 162.67 ± 44.98 | 177.76 ± 55.93 |
| | week-8 | 139.22 ± 89.05 | 159.41 ± 73.02 |
| HDL cholesterol | week-0 | 42.97 ± 9.39 | 41.54 ± 26.79 |
| (mg/dL) | week-8 | 58.33 ± 28.57* | 46.47 ± 24.95* |

**p < 0.05;
*p < 0.001;
***p < 0.0001 compared to week-0

Table 8 below shows the effects of hydroxycitrate (HCA) drink on some oxidative stress, hepatic and renal toxicity markers after eight weeks of treatment.

TABLE 8

| | | HCA (A) | HCA (B) |
|---|---|---|---|
| Plasma antioxidant | week-0 | 402.22 ± 33.71 | 447.77 ± 34.90 |
| capacity (μmol) | | | |
| Eq catechin/L | week-8 | 487.90 ± 42.13* | 386.66 ± 60.76 |
| MDA (μmol) | week-0 | 2.04 ± 0.43 | 2.31 ± 0.39 |
| | week-8 | 2.88 ± 0.32 | 2.58 ± 0.51* |
| ASAT (UI) | week-0 | 67.03 ± 13.42 | 85.57 ± 18.71 |
| | week-8 | 56.44 ± 10.04* | 49.34 ± 10.71* |
| ALAT (UI) | week-0 | 19.16 ± 3.97 | 12.11 ± 3.87 |
| | week-8 | 43.81 ± 7.89** | 38.19 ± 7.19** |
| Creatinin (mg/dL) | week-0 | 4.43 ± 0.91 | 2.80 ± 0.89 |
| | week-8 | 6.87 ± 0.86* | 2.9 ± 0.8*** |

**p < 0.05;
*p < 0.001;
***p < 0.0001 compared to week-0

| | | | Standard | Average Standard |
|---|---|---|---|---|
| | HCA | Average | Deviation | Error |
| BMI0-8 | A | −1.6323 | 2.34364 | .35332 |
| | B | −1.4875 | 3.02632 | .45624 |
| BMI0-2 | A | −1.6323 | 2.34364 | .35332 |
| | B | −1.4875 | 3.02632 | .45624 |
| FAT0-8 | A | 1.3054* | 3.79254 | .55918 |
| | B | 1.6852 | 3.07012 | .46284 |
| FAT0-2 | A | −.2675 | 2.47988 | .36564 |
| | B | −1.9564 | 3.45383 | .52068 |

*p < 0.01 compared with HCA (B)

Accordingly, it should be appreciated that the present disclosure is directed to methods for increasing weight loss and improving related health factors in a mammal. It should also be appreciated that the Garcinia indica fruit purée provided as part of the study demonstrates significantly more weight loss in mammals than the equivalent or even higher doses of HCA, alone or in combination with other ingredients. It is believed that the Garcinia indica fruit purée of the present invention provides highly bioavailable HCA in its natural form that works synergistically with the natural anyhocyanins found in Garcinia indica, specifically cyanidin-3-sambubioside and cyanidine 3-glucoside to provide statistically significant weight loss benefit demonstrated by the experiments discussed herein.

These data suggest a broad biological mechanism for cyanidin-3-sambubioside and cyanidine 3-glucoside as adjuvants to naturally occurring HCA (and not HCA salts) involving regulation of body weight, body composition and body metabolic rate.

In one embodiment of the present invention, a method for increasing weight loss in a mammal in need of such effect is provided. The method includes administering to a mammal an effective amount of a composition of Garcinia indica fruit purée.

In another embodiment of the present invention, a method for administering to a mammal an effective amount of a composition of naturally occurring HCA and anthocyanins from Garcinia indica fruit purée is provided.

In yet another embodiment of the present invention, a method of administering to a mammal an effective amount of a composition of naturally occurring HCA, cyanidin-3-sambubioside and cyanidine 3-glucoside from Garcinia indica fruit purée is provided.

In one aspect of the present invention, 1000-2000 milligrams of HCA and 300-1200 milligrams of anthocyanins from Garcinia indica fruit purée are provided to the mammal daily.

In another aspect of the present invention, 500 ml of Garcinia indica fruit purée is provided to the mammal daily.

In yet another aspect of the present invention, the effective amount of the composition decreases fat in the mammal.

In yet another aspect of the present invention, the effective amount of the composition decreases systolic and diastolic blood pressure in the mammal.

In yet another aspect of the present invention, the effective amount of the composition decreases triglyceride levels in the mammal.

In yet another aspect of the present invention, the effective amount of the composition decreases blood glucose levels in the mammal.

In yet another aspect of the present invention, the effective amount of the composition decreases total cholesterol levels in the mammal.

In yet another aspect of the present invention, the effective amount of the composition increases HDL cholesterol levels in the mammal.

In yet anther embodiment, a method of making a stabilized naturally occurring hydroxycitric acid composition is provided. The method comprises extracting Garcinia fruit containing hydroxycitric acid with an aqueous liquid comprising at least 8 mg/ml anthocyanin per 100 ml of liquid, to provide an aqueous extract and mixing the aqueous extract with a liquid quaternizing agent to yield a quaternizing agent extract that contains 1000 mg to 2500 mg of naturally occurring hydroxycitric acid.

In yet another aspect of the present invention, the quaternizing agent is a trialkylamine comprising an alkyl chosen from the group consisting of octyl, caprylyl, isooctyl, lauryl and decyl.

In yet another aspect of the present invention, the trialkylamine is tricaprylylamine.

In yet another aspect of the present invention, an anthocyanin-stabilized hydroxycitric acid composition prepared according to the methods described herein is provided.

In yet another aspect of the present invention, the stabilized naturally occurring hydroxycitric acid composition described herein is mixed with an edible food material.

In yet another aspect of the present invention, the stabilized naturally occurring hydroxycitric acid composition described herein is mixed with a drinkable liquid.

In yet another aspect of the present invention, the stabilized naturally occurring hydroxycitric acid composition described herein is mixed with an excipient. In yet another embodiment of the present invention, a method of reducing body weight comprising administering to a mammal in need of such weight reduction an effective amount of the compositions described herein are provided.

### REFERENCES

Clouatre, D, Rosenbaum, M. The diet and health benefits of HCA. A Keats Good Health Guide. pp. 9, 1994.

Sergio W. A natural food, the malabar tamarind, may be effective in the treatment of obesity. Med Hypotheses 1988;27:39-40.

Sullivan A C, Triscari J, Hamilton J G, Miller O N. Effect of (−)-hydroxycitrate upon the accumulation of lipid in the rat. II. appetite. Lipids 1974;9:129-34.

Sullivan A C, Triscari J, Cheng L. Appetite regulation by drugs and endogenous substances. Curr Concep Nutr 1983;12:139-67.

Lowenstein J M. Effect of (−)-hydroxycitrate on fatty acid synthesis by rat liver in vivo. J Biol Chem 1971;246:629-32.

Triscari J, Sullivan A C. Anti-obesity activity of a novel lipid synthesis inhibitor. Int J Obes 1984;8:227-39.

Sullivan A C, Triscari J, Hamilton J G, Miller O N, Wheatley V R. Effect of (−)-hydroxycitrate upon the accumulation of lipid in the rat. I. lipogenesis. Lipids 1974;9:121-8.

Ramos R R, Saenz J L S, Aguilar R J A. Extract of Garcinia cambogia in controlling obesity. Investigacion Medica Internacional 1995;22:97-100.

Bagchi, D., Deshmukh, N. S., Soni, M. G., Bagchi, M., 2007. Safety of a novel calcium/potassium salt of (−)-hydroxycitric acid: I. Two generation reproduction toxicity study. EuroTox. 44th Congress. Abstract No.: F03.

CSIR (1956). Garcinia. Council of Scientific and Industrial Research, India. The wealth of India: a dictionary of Indian raw materials and industrial products. Raw materials, Vol. IV. pp. 99-108.

DiNovi, M. J., Kuznesof, P. M., 1995. Estimating Exposure To Direct Food Additives And Chemical Contaminants in the Diet. Available at http://www.cfsan.fda.gov/˜dms/opa-cg8.html.

DSHEA, 1994. Dietary Supplements Health and Education Act of 1994. US Food and Drug Administration, Center for Food Safety and Applied Nutrition, Washington, D.C.

FASEB, 1988. Estimation of Exposure to Substances in the Food Supply, Anderson, S. A., Editor, FDA Contract No. 223-84-2059, Life Sciences Research Office, Bethesda, Md.

Hedrick, U. P., 1919. Garcinia indica. In: Sturtevant's Notes on Edible Plants. Library of Congress Catalog Number: 76-184690. Dover Publication Inc., New York, pp. 285-286.

Jayaprakash, G. K., Sakariah, K. K., 2002. Determination of organic acids in leaves and rinds of Garcinia indica (Desr.) by HPLC. J. Pharm. Biomed. Anal. 28, 379-384.

Jena, B. S., Jayaprakasha, G. K., Singh, R. P., Sakariah, K. K., 2002. Chemistry and biochemistry of (−)-hydroxycitric acid in commercial samples of Garcinia cambogia extracts by liquid chromatography using ultraviolet detection. Journal of Liquid Chromatography Related Technology 23, 915-923.

Khandare, A. L., Rao, G. S., Lakshmaiah, N., 2002. Effect of tamarind ingestion on fluoride excretion in humans. Eur. J. Clin. Nutr. 56, 82-85.

Kirtikar, K. R., Basu, B. D., 1984. In: Indian Medicinal Plants. Vol. I, Blatter, E., Caius, J. F. and Mhaskar, K. S., Editors, Allahabad, India.

Krishnamurthy, N., Lewis, Y. L., Ravindranath, B., 1982. Chemical constituents of Kokam fruit rind. J. Food Sci. Technol. 19, 97-100.

Kruijsen, F., 2008. Garcinia indica (kokum) from south India. Benefiting from the potential of underutilized fruits. International Tropical Fruits Network. Article available at: http://www.itfnet.org/contents/selectedArticals/ITFnet_Articals01.pdf.

Majeed, M., Badmaev, V. and Rajendran, R., 1998. Potassium hydroxycitrate for the suppression of appetite and induction of weight loss. Assignee: Sabinsa Corporation (Piscataway, N.J.). U.S. Pat. No. 5,783,603. Mar. 31, 1997. US Patent and Trademark Office, Washington D.C.

Martin, F. W., Campbell, C. W., Ruberte, R. M., 1987. Perennial edible fruits of the tropics: an inventory. United States Department of Agriculture, Agricultural Research Service, Washington, D.C., p. 212.

Martius, C., Maue, R., 1941. Preparation, physiological behavior, and importance of hydroxycitric acid and its isomers. Zeitschrift fur Physiological Chemistry 269, 33-39.

Mathew, K. S., 1997. The Portuguese and the study of medicinal plants in India in the sixteenth century. Indian J. Hist. Sci. 32, 369-376.

Mattes, D. R., Bormann, L., 2000. Effects of (−)-hydroxycitric acid on appetitive variables. Physiology and Behavior 71, 87-94.

MRCA, 1965. Market Research Corporation of America (MRCA), in conjunction with the “Food intake and nutritive value of the diets of men, women and children in United States, Spring 1965”. A Preliminary Report by the Consumer and Food Economics Research Division, Agricultural Research Division. United States Department of Agriculture, Washington, D.C.

Ohia, S. E., Opere, C. A., LeDay, A. M., Bagchi, M., Bagchi, D., Tohs, S. J., 2002. Safety and mechanism of appetite suppression by a novel hydroxycitric acid extract (HCA-SX). Molecular and Cellular Biochemistry 238, 89-103.

Peter, K. V., 2001. Handbook of Herbs and Spices. CRC Press, Boca Raton, Fla., USA.

Pruthi, J. S., 2001. Kokam. In: Spices and Condiments. National Book Trust, India. pp. 162-165.

Raju, V. K., Reni, M., 2001. Kokam and Cambodge. In: Handbook of Herbs and Spices. Editor-Peter, K. V., 2nd Edition. CRC Press, pp 207-215.

Sampathu, S. R., Krishnamurthy, N., 1982. Processing and utilisation of Kokam (Garcinia indica). Indian Spices 19, 15-16.

Saito, M., Ueno, M., Ogino, S., Kubo, K., Nagata, J., Takeuchi, M., 2005. High dose of Garcinia cambogia is effective in suppressing fat accumulation in developing male Zucker obese rats, but highly toxic to the testis. Food and Chemical Toxicology 43, 411-419.

Shara, M., Ohia, S. E., Schmidt, R. E., Yasmin, T., Zardetto-Smith, A., Kincaid, A., Bagchi, M., Chatterjee, A., Bagchi, D., Stohs, S. J., 2003a. Dose- and time-dependent effects of a novel (−)-hydroxycitric and extract on body weight, hepatic and testicular lipid peroxidation, DNA fragmentation and histopathological data over a period of 90-days. Molecular and Cellular Biochemistry 254, 339-346.

Shara, M., Ohia, S. E., Stohs, S. J., 2003b. 90-Day oral toxicity of super CitriMax in male and female Sprague-Dawley rats. Toxicology Research Center, Creighton University Medical Center, Omaha, Nebr., Feb. 21, 2003, ARC#0598, pp. 1-134.

Shara, M., Ohia, S. E., Schmidt, R. E., Yasmin, T., Zardetto-Smith, A., Kincaid, A., Bagchi, M., Chatterjee, A., Bagchi, D., Stohs, S. J., 2004. Physico-chemical properties of a novel (−)-hydroxycitric acid extract and its dose- and time-dependent effects on body weight, selected organ weights, hepatic lipid peroxidation and DNA fragmentation, hematology and clinical chemistry, and histopathological changes over a period of 90-days. Molecular and Cellular Biochemistry 260, 171-186.

Soni, M. G., Burdock, G. A., Preuss, H. G., Stohs, S. J., Ohia S. E., Bagchi, D., 2004. Safety assessment of (−)-hydroxycitric acid and Super CitriMax, a novel calcium/potassium salt. Food and Chemical Toxicology 42, 1513-1529.

Unibar Corporation (2008). Information on description, specifications, composition and manufacturing of Garcinia indica fruit extract.

USDA., 2000. 1994-1996, 1998 Continuing Survey of Food Intakes by Individuals (CSFII) and Diet and Health Knowledge Survey (DHKS). U.S. Department of Agriculture (USDA); Riverdale, Md.

Watt, G., 1890. Garcinia indica. In: A Dictionary of Economic Products of India. Vol. III., Dacrydium to Gordonia. W.H. Allen & Co., London. pp. 466-469.

Wildman, R. E. C., Handbook of Neutraceuticals and Functional Foods. CRC Press, Boca Raton, Fla., USA.

Yamada, T., Hida, H., Yamada, Y., 2007. Chemistry, physiological properties, and microbial production of hydroxycitric acid. Applied Microbiology and Biotechnology. 75, 977-82.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method for increasing weight loss in a mammal in need of such effect, the method comprising:
administering to a mammal a weight loss effective amount of Garcinia indica fruit purée.

2. The method of claim 1, wherein the effective amount of Garcinia indica fruit purée is 500 ml to 1000 ml daily.

3. The method of claim 1, wherein the effective amount also decreases fat in the mammal.

4. The method of claim 1, wherein the effective amount also decreases systolic and diastolic blood pressure in the mammal.

5. The method of claim 1, wherein the effective amount also decreases triglyceride levels in the mammal.

6. The method of claim 1, wherein the effective amount also decreases blood glucose levels in the mammal.

7. The method of claim 1, wherein the effective amount also decreases total cholesterol levels in the mammal.

8. The method of claim 1, wherein the effective amount provides natural HCA and anthocyanins that are more bioavailable and cause fewer side effects than HCA salts to the mammal.

9. A method for increasing weight loss in a mammal in need of such effect, the method comprising:
administering to a mammal a weight loss effective amount of naturally occurring HCA, cyanidin-3-sambubioside and cyanidine 3-glucoside from Garcinia indica fruit purée.

10. The method of claim 9, wherein the effective amount is 1000 mg to 2000 mg milligrams of naturally occurring HCA and 50 mg to 500 mg of cyanidin-3-sambubioside and cyanidine 3-glucoside from Garcinia indica fruit purée daily.

11. The method of claim 10, wherein the effective amount also decreases fat in the mammal.

12. The method of claim 9, wherein the effective amount also decreases systolic and diastolic blood pressure in the mammal.

13. A method of making a stabilized naturally occurring hydroxycitric acid composition, comprising:
extracting Garcinia fruit containing hydroxycitric acid with an aqueous liquid comprising at least 8 mg/ml anthocyanin per 100 ml of liquid, to provide an aqueous extract; and
mixing the aqueous extract with a liquid quaternizing agent to yield a quaternizing agent extract that contains 1000 mg to 2500 mg of naturally occurring hydroxycitric acid.

14. The method of claim 13, wherein the quaternizing agent is a trialkylamine comprising an alkyl chosen from the group consisting of octyl, caprylyl, isooctyl, lauryl and decyl.

15. The method of claim 14, wherein the trialkylamine is tricaprylylamine.

16. An anthocyanin-stabilized hydroxycitric acid composition prepared according to the method of claim 13.

17. A food comprising the composition of claim 16 mixed with an edible food material.

18. A drink comprising the composition of claim 16 mixed with a drinkable liquid.

19. A dietary supplement comprising the composition of claim 16 mixed with an excipient.

20. A method of reducing body weight comprising administering to a mammal in need of such weight reduction an effective amount of the composition of claim 16.
